(19) United States
(12) Patent Application Publication
(10) Pub. No.: US 2013/0090665 A1
(43) Pub. Date: Apr. 11, 2013

(54) Tonsil Debris Removal

(71) Applicants: Jason D. Linde, Dobbs Ferry, NY (US); Stacey L. Silvers, New York, NY (US)

(72) Inventors: Jason D. Linde, Dobbs Ferry, NY (US); Stacey L. Silvers, New York, NY (US)

(21) Appl. No.: 13/435,886

(22) Filed: Mar. 30, 2012

Related U.S. Application Data
(60) Provisional application No. 61/469,422, filed on Mar. 30, 2011.

(51) Int. Cl. A61B 17/24

(52) U.S. Cl. CPC A61B 17/24
USPC 606/106

(57) ABSTRACT

A solution for removing debris from an individual's tonsil, such as loose and embedded tonsil stones and tonsil debris.

### REFERENCE TO RELATED APPLICATIONS

The current application claims the benefit of co-pending U.S. Provisional Application No. 61/469,422, titled “Tonsil Debris Removal,” which was filed on 30 Mar. 2011, and which is hereby incorporated by reference.

### TECHNICAL FIELD

The disclosure relates generally to the removal of debris from a tonsil, and more particularly, to a device for removing debris from the tonsil.

### BACKGROUND ART

Tonsils can collect debris, often referred to as tonsilloliths or tonsil stones. Several approaches have been proposed for removing debris from a tonsil. These approaches include irrigation, scraping, gargling, or surgical excision. One approach proposes the use of a medicine dropper, such as one with a curved tip, to suck out sufficiently small tonsil debris. Another approach seeks to dissolve the tonsil debris through the use of nasal drops and a spray. Other approaches seek to prevent the debris from collecting in the tonsil, e.g., by resurfacing the tonsil using a laser, removing the tonsils, adjusting an individual's diet, and/or the like.

### SUMMARY OF THE INVENTION

Aspects of the invention provide a solution for removing debris from an individual's tonsil, such as loose and embedded tonsil stones and tonsil debris.

A first aspect of the invention provides a device for removing tonsil debris, the device comprising: a tip including an aperture; a debris manipulation structure attached to the tip and located adjacent the aperture; a suction device; and a hollow arm fluidly connecting the tip and the suction device.

A second aspect of the invention provides a kit for removing tonsil debris, the kit comprising: a suction device; a set of hollow arms, each hollow arm having a first end configured to be attached adjacent to the suction device and a second end; a set of tips, each tip including a first end configured to be attached to the second end of each of the hollow arms and a second end including an aperture; and a debris manipulation structure, wherein the debris manipulation structure is one of: permanently attached or configured to be temporarily attached to at least one of the set of tips in a location adjacent to the aperture.

A third aspect of the invention provides a method of removing tonsil debris, the method comprising: locating an aperture of a tip including a debris manipulation structure located adjacent thereto at a location of the tonsil debris; and operating a suction device in fluid connection with the tip and the debris manipulation structure to remove the tonsil debris.

The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIG. 1 shows an illustrative device for removing tonsil debris according to an embodiment.

FIG. 2 shows a second illustrative device for removing tonsil debris according to an embodiment.

FIG. 3 shows a third illustrative device for removing tonsil debris according to an embodiment.

FIG. 4 shows a fourth illustrative device for removing tonsil debris according to an embodiment.

FIG. 5 shows front and side views of three illustrative tips according to an embodiment.

FIG. 6 shows an illustrative kit according to an embodiment.

It is noted that the drawings may not be to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As indicated above, aspects of the invention provide a solution for removing debris from an individual's tonsil, which can be more effective than previous approaches. As used herein, unless otherwise noted, the term “set” means one or more (i.e., at least one) and the phrase “any solution” means any now known or later developed solution.

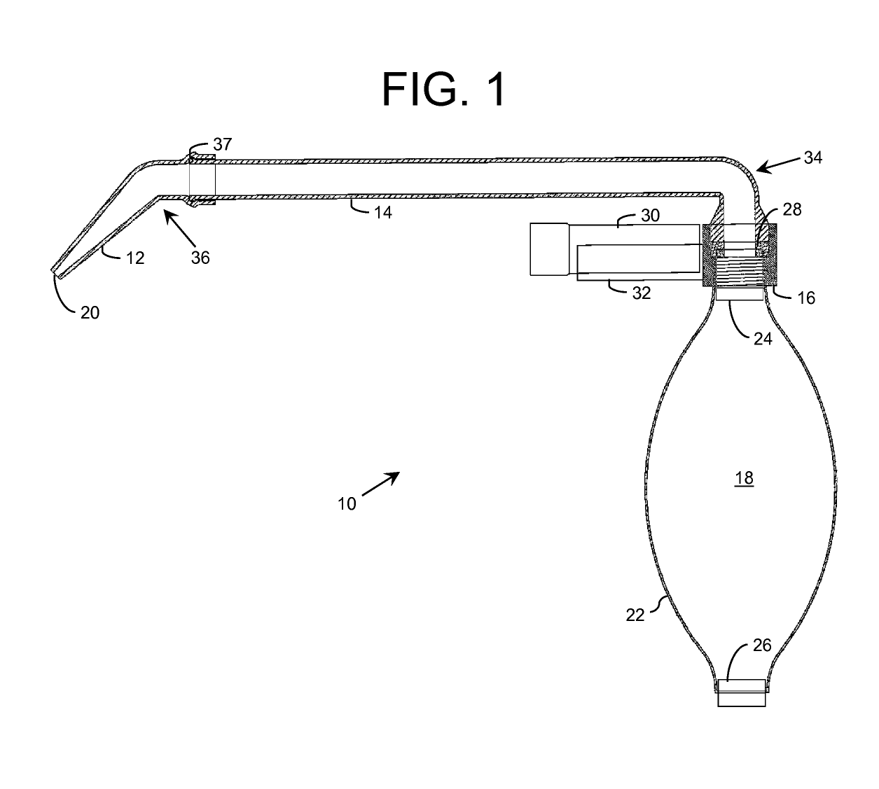

Turning to the drawings, FIG. 1 shows an illustrative device 10 for removing tonsil debris (e.g., tonsillolith or tonsil stone) according to an embodiment. As illustrated, the device 10 includes a hollow tip 12 having an aperture 20 at one end, a hollow arm 14, a central mechanism 16 (e.g., an armature or clip), and a suction device 18. The hollow tip 12, hollow arm 14, central mechanism 16, and suction device 18 can be sequentially attached to one another to provide a fluid (e.g., air or liquid)-tight passage between the suction device 18 and the aperture 20 at the end of the hollow tip 12. Each connection can be made using any permanent or releasable solution for forming a fluid-tight connection between two components including, for example, glue, a screw/thread, a snap fit, a flange, and/or the like. Furthermore, it is understood that two or more of the components of the device 10 can be formed using a single structure (e.g., a molded piece). For example, two or more of the tip 12, the arm 14, and/or the central mechanism 16 can comprise a single structure. It also is understood that an embodiment of the device 10 can be implemented without the central mechanism 16, in which case the arm 14 can be directly attached to the suction device 18.

During operation of the device 10, a user can direct the aperture 20 at the end of the tip 12 to a location adjacent to an individual's tonsil, and in particular, adjacent to a location of tonsil debris, which is desired to be removed. When located in a desired position, the user can operate the suction device 18 to cause a pressure differential at the aperture 20, which can create suction to assist in removing the tonsil debris. While the suction device 18 is shown including a compressible bulb 22 (e.g., a latex bulb), it is understood that any type of manual or automated device for generating a pressure differential at the aperture 20 can be utilized.

Regardless, the suction device 18 can enable a user to provide adequate suction during operation of the device 10 while allowing the user to maintain a steady hand during the extraction of tonsil debris. In an embodiment, the suction device 18 includes an integrated intake valve 24 and exhaust valve 26. In this case, when the user squeezes the compressible bulb 22, the exhaust valve 26 can open allowing the passage of a fluid (e.g., air) out of the compressible bulb 22, while the intake valve 24 remains closed. Conversely, when the user releases the compressible bulb 22, the intake valve 24 can open allowing the passage of a fluid (e.g., air) into the compressible bulb 22, while the exhaust valve 26 remains closed. Each of the intake valve 24 and the exhaust valve 26 can be implemented using any type of valve, such as a pivot valve. While such a compressible bulb 22 may provide a lower pressure than other types of suction devices, a user can be able to more accurately place the tip 12 adjacent to the tonsil debris, thereby reducing the requirement for higher pressure.

In an embodiment, the intake valve 24 is located adjacent to an opposing end of the arm 14 from the tip 12, and is configured to prevent fluid from flowing into the arm 14 and exiting the aperture 20 during compression of the compressible bulb 22, while allowing fluid to pass through the aperture 20 and through the arm 14 during decompression of the compressible bulb 22. Such a configuration allows successive pumping of the compressible bulb 22 without causing fluid to flow out of the aperture 20, which may be located within the individual's mouth. As a result, the user can operate the suction device 18 a plurality of times without removing the tip 12 from a location including tonsil debris, such as a tonsil cavity, between successive operations.

In an embodiment, the device 10 also includes a filter 28, which is located between the aperture 20 and the interior of the suction device 18 (e.g., the interior of the compressible bulb 22). For example, the filter 28 can be located within the central mechanism 16 between the arm 14 and the suction device 18. Alternatively, the filter 28 can be incorporated into the suction device 18, e.g., adjacent to the intake valve 24. The filter 28 can comprise any type of filter, screen, or the like, which can prevent debris of a minimum size from entering the aperture 20 and traveling into the suction device 18. The filter 28 can be formed of any material, and can be reusable (e.g., washable), disposable, and/or the like. In an embodiment, the filter 28 comprises a stainless steel screen with openings of any desired size. Furthermore, the filter 28 can be temporarily or permanently placed within the central mechanism 16 or suction device 18. When permanently placed within the central mechanism 16, the device 10 can be configured to enable the central mechanism 16, including the filter 28, to be washed after use of the device 10.

In an embodiment, the device 10 also includes a light source 30, which is configured to direct light onto an area adjacent to the aperture 20 on the tip 12 and is attached to another component of the device 10 using any attachment mechanism. For example, the light source 30 can comprise a light emitting diode (LED) light source, which is attached to the central mechanism 16 using any solution. However, it is understood that the light source 30 can utilize any solution for generating and/or directing light. In an embodiment, the attachment mechanism comprises a housing 32 located on/affixed to the central mechanism 16 within which the light source 30 can be inserted and held in place, e.g., by a set of clips, screw/thread, or the like. In this case, the attachment mechanism can hold the light source 30 in a fixed location, and light generated by the light source 30 will be directed in a fixed direction. In another embodiment, the arm 14 and/or tip 12 can include a light directing structure, such as one or more optical fibers, which can direct light from a light source, e.g., located on central mechanism 16, to a location adjacent to the aperture 20.

As illustrated, the arm 14 can include a bend 34 on an opposing side of the arm 14 as the tip 12. The bend 34 can be a substantially right angle, thereby locating the central mechanism 16 and the suction device 18 substantially perpendicular to the arm 14. In this manner, the line of sight of a user utilizing the device 10 is not impeded by the components of the device 10 that remain external of the mouth, such as the suction device 18 and the central mechanism 16. Furthermore, the aperture 20 of the tip 12 can be located at an angle relative to the direction of the arm 14. To this extent, the tip 12 and/or the arm 14 can include a second bend 36. In an embodiment, the arm 14 and tip 12 can form an angle of approximately forty-five degrees.

In a further embodiment, the arm 14 and tip 12 are connected using a connection mechanism 37 which allows the aperture 20 of the tip 12 to be located at any of a plurality of positions about an axis of the arm 14. For example, the arm 14 and tip 12 can be connected using a connection mechanism 37 that allows the tip 12 to be rotated with respect to the arm 14 (e.g., a pivotable connection mechanism, a circular male/female connection mechanism, and/or the like). Similarly, the arm 14 and tip 12 can be connected using a connection mechanism 37 that allows the tip 12 to be located in any of a plurality of fixed locations about the axis (e.g., a toothed connection mechanism, a polygonal male-female connection mechanism, and/or the like). In an embodiment, the connection mechanism 37 comprises a circular snap fit connection mechanism, in which the tip 12 snaps into place over a bump located on the end of the hollow arm 14 to assist in keeping the tip 12 attached to the hollow arm 14 during use, while allowing the tip 12 to be rotated about the axis of the hollow arm 14.

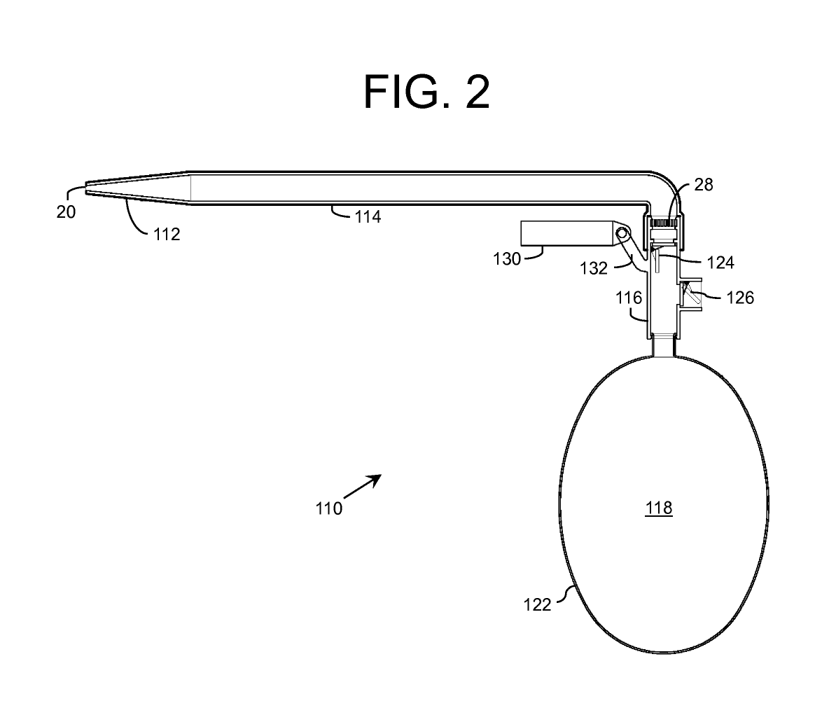

It is understood that the device 10 is only illustrative of various device configurations that can be utilized according to embodiments. For example, FIG. 2 shows a second illustrative device 110 for removing tonsil debris according to an embodiment. In device 110, the central mechanism 116 includes both the intake valve 124 and the exhaust valve 126 located thereon. As a result, the suction device 118 can comprise a compressible bulb 122, without any valves integrated thereon. Furthermore, the light source 130 is shown attached to the central mechanism 116 using a rotatable attachment mechanism 132 (e.g., a bracket with a pivot attachment). In this case, a user can redirect the light emitted from the light source 130 to a desired location using any solution (e.g. the pivot attachment(s)). Additionally, the tip 112 is shown being substantially coplanar with the hollow arm 114.

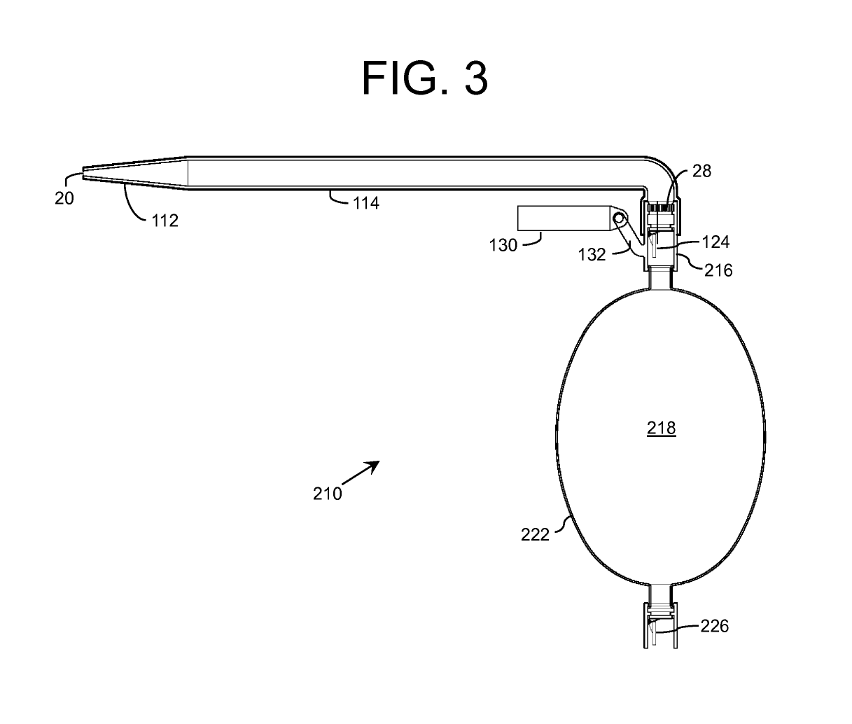

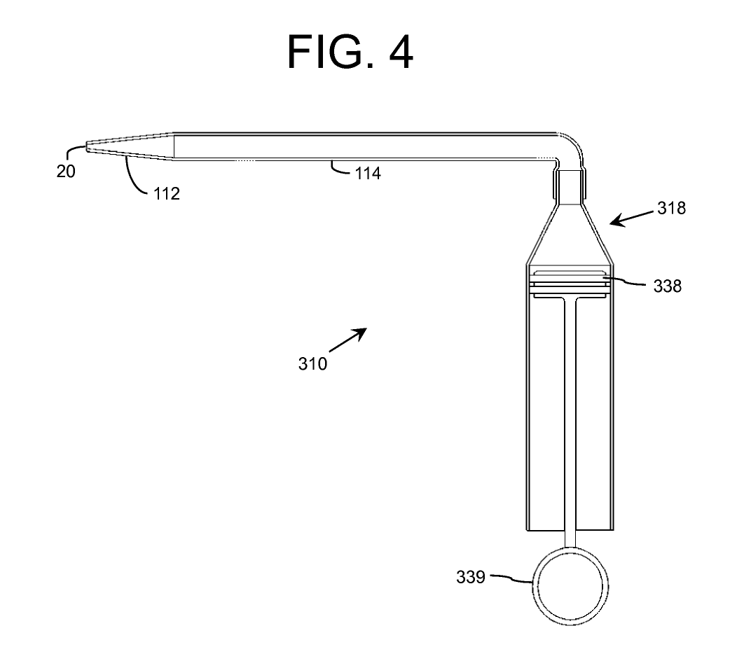

FIG. 3 shows a third illustrative device 210 for removing tonsil debris according to an embodiment. In device 210, the central mechanism 216 includes the intake valve 124, while the exhaust valve 226 is located on an opposing end of the compressible bulb 222 of the suction device 218. FIG. 4 shows a fourth illustrative device 310 for removing tonsil debris according to an embodiment. In device 310, the suction device 318 comprises a piston type mechanism, in which a piston seal 338 can be pulled down within a piston sleeve in order to create suction at the aperture 20 using a finger pull 339. While not shown, it is understood that the device 310 can be configured to include various components similar to the other embodiments (e.g., valves, filter, light, and/or the like).

It is understood that each of the various components of the illustrative devices shown and described herein can be manufactured from any material. Illustrative materials include: stainless steel, polycarbonate, polystyrene, a thermoplastic, such as acrylonitrile-butadiene-styrene (ABS) plastic, polyvinyl chloride (PVC), and/or the like. In an embodiment, one or more components, such as a tip, arm, central mechanism, and/or a compressible bulb described herein, is manufactured using an at least partially transparent material, such as a transparent rubber or plastic material. Use of the at least partially transparent material can assist a user in cleaning the various components after use. The various components can have any dimensions that will be sufficient to allow for ready use of the device to access a location of the tonsils within an individual's mouth. In an embodiment, the tip 12 and arm 14 can have a total length between approximately 5-7 inches. The arm 14 can have an interior diameter of approximately a quarter of an inch, while the aperture 20 on the tip 12 can have an interior diameter of approximately one-twentieth of an inch. However, it is understood that these dimensions are only illustrative of various possible dimensions.

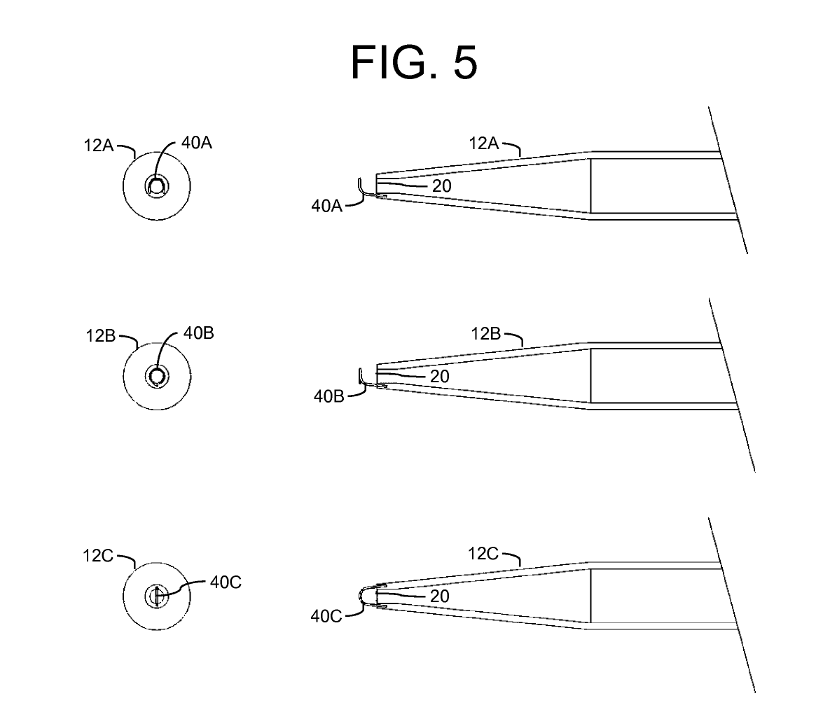

It is understood that various configurations of tips 12, 112 can be utilized. In an embodiment, a shape of the aperture 20 of the tip 12, 112 or a structure located adjacent thereto is configured to produce natural surface tension, which can assist the suction being applied through the aperture 20 of the tip 12 in drawing the debris from a cavity of the tonsil. Furthermore, the tip can be configured to reduce a tendency for the tip to vacuum itself to the tonsil rather than the debris particle, e.g., by having a shape/structure which allows the tip to sit against a bottom of a cavity including the debris. For example, FIG. 5 shows front (left side of the figure) and side (right side of the figure) views of three illustrative tips 12A-12C according to an embodiment. Each tip 12A-12C includes a structure 40A-40C, respectively, located adjacent to the respective apertures 20. Each structure 40A-40C can be temporarily or permanently attached to the corresponding tip 12A-12C using any solution. In an embodiment, each structure 40A-40C is formed of a metal, such as stainless steel. However, various structures can be formed as part of the tip (e.g., part of a molded plastic tip).

With respect to tip 12A, the structure 40A includes a loop, which is attached to the tip 12A in two locations next to the aperture 20. The loop extends a small distance from the aperture 20 along a plane substantially perpendicular to the aperture 20 before curling up forming an upside down “u” on a plane substantially parallel with the aperture 20. With respect to tip 12B, the structure 40B is attached to the tip 12B at a single location next to the aperture. The structure 40B extends a small distance from the aperture 20 along a plane substantially perpendicular to the aperture 20 before curling up forming a circle on a plane substantially parallel with the aperture 20. With respect to tip 12C, the structure 40C is attached to the tip 12C in two locations on opposing sides of the aperture 20 with a loop formed there between, which is on a plane substantially perpendicular to the aperture 20. It is understood that structures 40A-40C are only illustrative of various types of structures, which can be incorporated adjacent to the aperture 20.

An embodiment provides a method for removing debris from a tonsil. In the method and using the device 10 of FIG. 1 as an illustrative device, a user (e.g. the individual with the tonsil debris or another individual) locates the aperture 20 of the tip 12 adjacent to debris lodged in the tonsil, e.g., with the use of the light source 30. The user can operate the suction device 18 (e.g., by compressing and releasing the compressible bulb 22 to create suction at the aperture 20, which can dislodge and/or remove the tonsil debris. For debris that is difficult to remove, the user can use the tip 12 (or a structure 40A-40C (FIG. 5) located adjacent to the aperture 20) to create surface tension on the debris and/or tonsil to assist in exposing and/or removing the debris.

Additionally, a user can utilize a device described herein to direct an irrigating fluid onto the tonsil/debris to assist in the removal of the debris. In an embodiment, a suction device 18 and/or valves 24, 26 associated therewith can be reconfigured and operated to perform irrigation of the tonsil and/or tonsil debris. For example, the device 10 shown in FIG. 1 can be reconfigured for irrigation by connecting the central mechanism 16 to the opposing side of the suction device 18. In this case, the valves 24, 26 on the suction device 18 work in the opposite manner as described above. The compressible bulb 22 and/or arm 14 can be filled with an irrigating liquid, such as water, a hydrogen peroxide solution, and/or the like, and squeezed to direct the irrigating liquid through the arm 14 and tip 12 and onto the tonsil and tonsil debris. Use of a hydrogen peroxide solution can assist in lifting debris via a foaming action created thereby. When multiple pumps of the compressible bulb 22 are required, the valve configuration causes the intake valve 24 to obtain fluid (e.g., air) from a location outside of the individual's mouth, thereby not allowing fluid from the individual's mouth to enter into the arm 14 displacing the irrigating fluid.

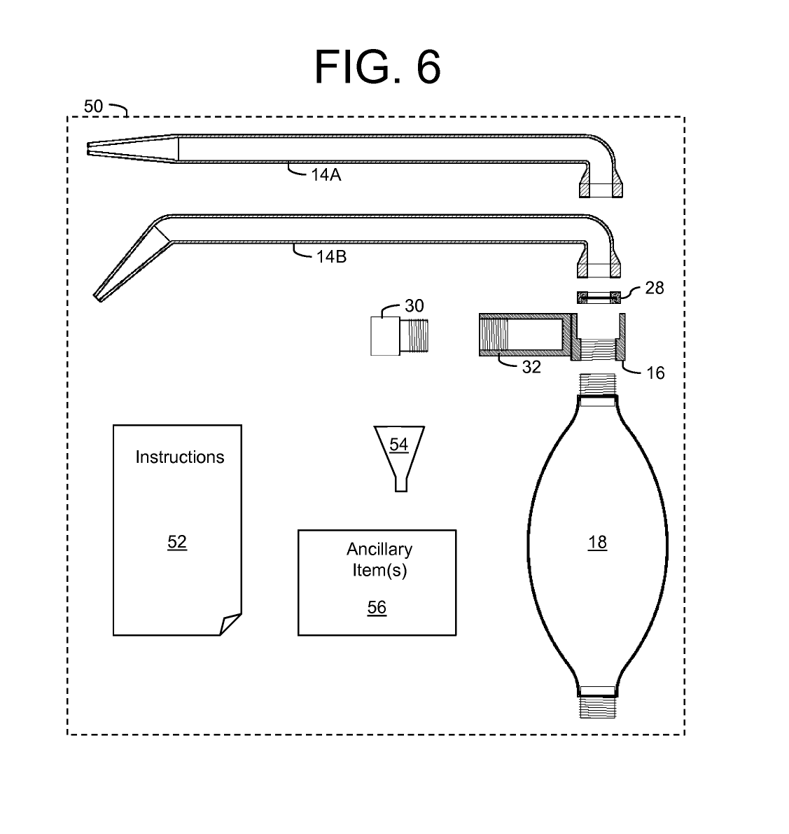

An embodiment provides a kit for removing debris from a tonsil. For example, FIG. 6 shows an illustrative kit 50 according to an embodiment. The kit 50 can include: a set of arms 14A, 14B, each of which is shown including an integrated tip; a suction device 18; a central mechanism 16, which includes a light housing 32; a light source 30; a set of filters 28; and/or the like. Furthermore, the kit 50 is shown including instructions 52, which can describe assembly, maintenance, and use of the device in a manner described herein. The kit 50 also can include one or more mechanisms, such as a funnel 54, for assisting in filling the arm 14A, 14B and/or suction device 18 with an irrigating liquid, a cleaning solution, and/or the like. Similarly, the kit 50 can include one or more ancillary items 56 for use in conjunction with the device, such as, for example, a cleaning device and/or a cleaning solution for use in cleaning one or more of the parts of the device, an irrigating solution, a mix for creating a cleaning or irrigating solution, and/or the like.

While the kit 50 is shown including a set of arms 14A, 14B, each with an integrated tip, it is understood that an embodiment of a kit can include a set of arms 14A, 14B each of which is configured to have a replaceable tip attached thereto. For example, an embodiment of a kit can include a first arm to which a replaceable tip can be attached in a planar fashion (e.g., as shown with respect to arm 14A), and a second arm having a bend adjacent to the attachment point of the replaceable tip, which will cause the tip to be located at an angle from the length of the arm (e.g., as shown with respect to arm 14B). In this case, the kit can include a set of replaceable tips, each of which can be configured differently. For example, the set of replaceable tips can include one or more of the tips 12A-12C shown in FIG. 5; one or more tips having apertures of different shapes (e.g., an angled aperture, a spoon/scoop-like projection from the aperture, a finger-like projection from the aperture, and/or the like); etc. Furthermore, the kit can include two or more tips configured in the same manner, but having different sized apertures 20.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A device for removing tonsil debris, the device comprising:
a tip including an aperture;
a debris manipulation structure attached to the tip and located adjacent the aperture;
a suction device; and
a hollow arm fluidly connecting the tip and the suction device.

2. The device of claim 1, wherein the debris manipulation structure is one of: a hook or a loop.

3. The device of claim 1, wherein the debris manipulation structure is substantially perpendicular to a plane of the aperture.

4. The device of claim 1, wherein the hollow arm includes a substantially perpendicular bend on an end of the hollow arm adjacent to the suction device.

5. The device of claim 1, wherein at least one of: the hollow arm or the tip includes a bend configured to locate the aperture on an angle with respect to an axis of the arm.

6. The device of claim 5, wherein the tip is connected to the hollow arm using a connection mechanism allowing the aperture of the tip to be located at any of a plurality of positions about the axis of the arm.

7. The device of claim 1, further comprising means for directing light adjacent to the aperture of the tip.

8. The device of claim 1, wherein the suction device is capable of being connected to the hollow arm in a first configuration for providing suction through the aperture and in a second configuration for expelling a fluid through the aperture.

9. The device of claim 1, further comprising a filter located between the aperture and the suction device.

10. The device of claim 1, further comprising a central mechanism including:
a first end attached to the hollow arm; and
a second end attached to the suction device.

11. The device of claim 1, wherein at least one of: the tip, the hollow arm, and the suction device is formed of an at least partially transparent material.

12. A kit for removing tonsil debris, the kit comprising:
a suction device;
a set of hollow arms, each hollow arm having a first end configured to be attached adjacent to the suction device and a second end;
a set of tips, each tip including a first end configured to be attached to the second end of each of the hollow arms and a second end including an aperture; and
a debris manipulation structure, wherein the debris manipulation structure is one of: permanently attached or configured to be temporarily attached to at least one of the set of tips in a location adjacent to the aperture.

13. The kit of claim 12, wherein the debris manipulation structure is one of: a hook or a loop.

14. The kit of claim 12, further comprising a light source, and means for mounting the light source adjacent to the suction device.

15. The kit of claim 12, wherein the suction device is capable of being connected to the hollow arm in a first configuration for providing suction through the aperture and in a second configuration for expelling a fluid through the aperture.

16. The kit of claim 12, further comprising a central mechanism including:
a first end attachable to the hollow arm; and
a second end attachable to the suction device.

17. The kit of claim 12, further comprising a set of filters for preventing debris from entering the suction device.

18. The kit of claim 12, further comprising instructions for operating a device for removing tonsil debris, the instructions including:
locating an aperture of a tip including a debris manipulation structure located adjacent thereto at a location of the tonsil debris; and
operating at least one of: the suction device or the debris manipulation structure to remove the tonsil debris.

19. A method of removing tonsil debris, the method comprising:
locating an aperture of a tip including a debris manipulation structure located adjacent thereto at a location of the tonsil debris; and
operating a suction device in fluid connection with the tip and the debris manipulation structure to remove the tonsil debris.

20. The method of claim 19, wherein the operating includes using the suction device to provide suction at the aperture.

21. The method of claim 19, wherein the operating includes using the suction device to expel a fluid through the aperture.
